(19) United States
(12) Patent Application Publication
(10) Pub. No.: US 2014/0275007 A1
(43) Pub. Date: Sep. 18, 2014

(54) OXAMAZIN ANTIBIOTICS

(71) Applicant: Rempex Pharmaceuticals, Inc., San Diego, CA (US)

(72) Inventors: Tomasz Glinka, Cupertino, CA (US); Scott Hecker, Del Mar, CA (US); Olga Rodny, Mill Valley, CA (US)

(21) Appl. No.: 13/829,304

(22) Filed: Mar. 14, 2013

(51) Int. Cl. C07D 205/085

(52) U.S. Cl. CPC C07D 205/085
USPC 514/210.15; 540/355

(57) ABSTRACT

Disclosed herein are oxamazin monobactam compounds and their use as antibiotics resistant to degradation by β-lactamases. Also disclosed are pharmaceutical compositions containing the compounds and methods of synthesis.

### BACKGROUND OF THE INVENTION

1. Field of the Invention

The present invention relates to the fields of chemistry and medicine. More particularly, the present invention relates to monobactam compounds that are useful for the treatment of bacterial infections, especially Gram-negative infections. The invention also relates to methods of using such compounds in the treatment of bacterial infections and to pharmaceutical compositions and pharmaceutical combinations containing such compounds.

2. Description of the Related Art

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of antibacterial therapy. The increase in antibiotic resistant strains has been particularly common in major hospitals and care centers. The consequences of the increase in resistant strains include higher morbidity and mortality, longer patient hospitalization, and an increase in treatment costs.

β-Lactam antibiotics have been widely used for the treatment of bacterial infections both in hospitals and in the general public. There are several classes of β-lactam antibiotics that have found clinical application; these include the penicillins, cephalosporins, cephamycins, carbacephems, oxacephems, carbapenems and monobactams.

The efficiency of all of these classes to cure bacterial infections has been impaired by the appearance of bacteria that are resistant towards the antibiotics. The prevalent cause of this resistance in Gram-negative bacteria is the expression by the bacteria of enzymes known as β-lactamases that are able to hydrolyse the β-lactam antibiotics rendering them inactive. Bacteria are able to produce a variety of β-lactamases, including penicillinases, cephalo sporinases, cephamycinases, carbapenemases, monobactamases, broad-spectrum β-lactamases and extended-spectrum β-lactamases.

Monocyclic β-lactam (monobactam) antibiotics inhibit mucopeptide synthesis in the bacterial cell wall, thereby blocking peptidoglycan crosslinking. They have a very high affinity for penicillin-binding protein-3 (PBP-3) and mild affinity for PBP-1a [Antimicrobial Agents and Chemotherapy (1983), 23(1), 98-104]. Monobactams have strong activity against susceptible gram-negative bacteria, including Pseudomonas aeruginosa. Monobactams are known to be effective against a wide range of bacteria including Citrobacter, Enterobacter, E. coli, Haemophilus, Klebsiella, Proteus, and Serratia species (Mosby's Drug Consult 2006 [16 ed.]. Mosby, Inc.)

Monocyclic β-lactam antibacterials received considerable attention [Chemotherapy (1987), 33(3), 165-171; Mayo Clinic proceedings (1991), 66(11), 1152-1157; Lindner, K. R., Bonner, D. P., & Koster, W. H. (2000). Monobactams. Kirk-Othmer Encyclopedia of Chemical Technology. John Wiley & Sons, Inc.] largely due to their specific resistance profiles against bacterial β-lactamase enzymes. For example, aztreonam and carumonam, two such antibiotics currently in use as antibacterials, show intrinsic resistance toward class B β-lactamases [Journal of Medical Microbiology (2008), 57(8), 974-979; Journal of Chemotherapy (2011), 23(5), 263-265] (zinc containing metalloenzymes). Activity against the broad spectrum of gram negative bacteria is a desirable characteristic of a modern β-lactam antibiotics and it is largely determined by the susceptibility of the β-lactam core to the enzymatic hydrolysis by a variety of bacterial β-lactamases. Thus, there is a need for new β-lactam antibiotics resistant to β-lactamases.

### SUMMARY OF THE INVENTION

The present invention relates to antimicrobial agents. Some embodiments include compounds, compositions, pharmaceutical compositions, use and preparation thereof. In particular, some embodiments, relate to monocyclic β-lactam compounds.

One embodiment disclosed herein includes a compound having the structure of Formula I:

[chemical structure]

In some embodiments of Formula (I):

R¹ and R² are independently selected from the group consisting of H, halide, CN, C₁₋₃ alkyl, —X(C₁₋₃ alkyl), —(C₁₋₃ alkyl)OH, —(C₁₋₃ alkyl)CN and C₁₋₃ haloalkyl, or alternatively R¹ and R² together with the atom to which they are attached form a three to four membered carbocyclic ring optionally substituted with one or more halide, or a four membered heterocyclic ring;

R³ and R⁴ are independently selected from the group consisting of halide, CN, C₁₋₃ alkyl, —X(C₁₋₃ alkyl), —X(C₁₋₃ haloalkyl), —(C₁₋₃ alkyl)OH, —(C₁₋₃ alkyl)CN and C₁₋₃ haloalkyl, or alternatively R³ and R⁴ together with the atom to which they are attached form a three to four membered carbocyclic ring optionally substituted with one or more halide, or a four membered heterocyclic ring;

R⁵ and R⁶ are independently selected from the group consisting of H, halide, CN, C₁₋₃ alkyl, —X(C₁₋₃ alkyl), —X(C₁₋₃ haloalkyl), —(C₁₋₃ alkyl)OH, —(C₁₋₃ alkyl)CN and C₁₋₃ haloalkyl, or alternatively R⁵ and R⁶ together with the atom to which they are attached form a three to four membered carbocyclic ring optionally substituted with one or more halide, or a four membered heterocyclic ring;

A is selected from the group consisting of C—H, C-halide, and N; and

each X is independently selected from O or S.

In addition to the foregoing, some embodiments include a pharmaceutical composition comprising a therapeutically effective amount of any one of the foregoing compounds and a pharmaceutically acceptable carrier, diluent, excipient or combination thereof.

In addition to the foregoing, some embodiments include any one of the foregoing compounds or compositions for the treatment of a bacterial infection.

In addition to the foregoing, some embodiments include methods for treating a bacterial infection comprising administering to a subject in need thereof, an effective amount of any one of the foregoing compounds or compositions.

Some embodiments further comprise administering an additional medicament.

In addition to the foregoing, some embodiments include the use of any one of the foregoing compounds or compositions in the preparation of a medicament for the treatment of a bacterial infection.

In some embodiments, the use of any one of the foregoing compounds or compositions, further comprises the use of an additional medicament for treating or preventing a bacterial infection.

In some embodiments, the additional medicament includes a β-lactamase inhibitor, efflux pump inhibitor, antifungal agent, an antiviral agent, an anti-inflammatory agent or an anti-allergic agent.

In some embodiments, the subject is a mammal.

In some embodiments, the mammal is a human.

In some embodiments, the infection comprises a bacteria that includes Pseudomonas aeruginosa, Pseudomonas fluorescens, Pseudomonas acidovorans, Pseudomonas alcaligenes, Pseudomonas putida, Stenotrophomonas maltophilia, Burkholderia cepacia group, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Acinetobacter anitratis, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtherias, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In some embodiments, the infection comprises a bacteria that includes Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

Some embodiments of the present invention include methods to prepare a compound of general Formula (I).

It is to be understood that both the foregoing general description and the following detailed description are exemplary and explanatory only and are not restrictive of the invention, as claimed.

### DETAILED DESCRIPTION OF THE INVENTION

The present invention relates to antimicrobial agents and potentiators thereof. Some embodiments include compounds, compositions, pharmaceutical compositions, uses thereof, including methods of preparation, and methods of treatment. In particular, the present invention relates to monocyclic β-lactam compounds. Some embodiments include compounds of Formula (I):

[chemical structure]

or pharmaceutically acceptable salts thereof.

In some embodiments, R¹ and R² are independently selected from the group consisting of H, halide, CN, C₁₋₃ alkyl, —(C₁₋₃ alkyl)CN, —(C₁₋₃ alkyl)OH, and C₁₋₃ haloalkyl, or alternatively R¹ and R² together with the atom to which they are attached form a three to four membered carbocyclic ring optionally substituted with one or more halide, or a four membered heterocyclic ring.

In some embodiments, R¹ and R² are H.

In some embodiments, R¹ and R² are independently selected from the group consisting of H, halide, CN, C₁₋₃ alkyl, —X(C₁₋₃ alkyl), —(C₁₋₃ alkyl)OH and haloalkyl.

In some embodiments, R¹ is H and R² is selected from the group consisting of halide, CN, C₁₋₃ alkyl, —(C₁₋₃ alkyl)CN, —(C₁₋₃ alkyl)OH, and C₁₋₃ haloalkyl. In some such embodiments, R² is selected from the group consisting of CN, CH₃, CH₂OH, CH₂CN and CH₂F.

In some embodiments, R¹ and R² are independently selected from the group consisting of halide and C₁₋₃ alkyl.

In some embodiments, R¹ and R² together with the atom to which they are attached form a three to four membered carbocyclic ring optionally substituted with one or more halide, or a four membered heterocyclic ring. In some such embodiments, R¹ and R² together with the atom to which they are attached form a ring selected from the group consisting of cyclobutyl, 3-fluoro-cyclobutyl, 2-oxetanyl, and 3-oxetanyl.

In some embodiments, R³ and R⁴ are independently selected from the group consisting of halide, CN, C₁₋₃ alkyl, —X(C₁₋₃ alkyl), —X(C₁₋₃ haloalkyl), —(C₁₋₃ alkyl)OH and haloalkyl.

In some embodiments, R³ and R⁴ are independently selected from the group consisting of C₁₋₃ alkyl and —(C₁₋₃ alkyl)OH.

In some embodiments, R³ and R⁴ are independently selected from the group consisting F, CH₃, CH₂F, CHF₂, CN, OCH₃, SCH₃, OCH₂F, SCH₂F, OCHF₂, SCHF₂, and CH₂CN.

In some embodiments, R³ and R⁴, R³ and R⁴ are independently selected from the group consisting F, CH₃, CH₂F, CHF₂, CN, OCH₃, SCH₃, OCH₂F, SCH₂F, OCHF₂, SCHF₂, and CH₂CN. In some such embodiments, R³ and R⁴ together with the atom to which they are attached form a ring selected from the group consisting of cyclobutyl, 3-fluoro-cyclobutyl, 2-oxetanyl, 3-oxetanyl, and cyclopropyl.

In some embodiments, R⁵ and R⁶ are independently selected from the group consisting of H, halide, C₁₋₃ alkyl, —X(C₁₋₃ alkyl), —X(C₁₋₃ haloalkyl), —(C₁₋₃ alkyl)OH, and C₁₋₃ haloalkyl, or alternatively R⁵ and R⁶ together with the atom to which they are attached form a three to four membered carbocyclic ring optionally substituted with one or more halide, or a four membered heterocyclic ring.

In some embodiments, R⁵ and R⁶ are H.

In some embodiments, R⁵ and R⁶ are independently selected from the group consisting of H, halide, CN, C₁₋₃ alkyl, —X(C₁₋₃ alkyl), —X(C₁₋₃ haloalkyl), —(C₁₋₃ alkyl)OH and haloalkyl.

In some embodiments, R⁵ is H and R⁶ is selected from the group consisting of halide, CN, C₁₋₃ alkyl, —X(C₁₋₃ alkyl), —X(C₁₋₃ haloalkyl), —(C₁₋₃ alkyl)CN, —(C₁₋₃ alkyl)OH, and C₁₋₃ haloalkyl. In some such embodiments, R⁶ is selected from the group consisting of F, CH₃, CH₂F, CHF₂, CN, OCH₃, SCH₃, OCH₂F, SCH₂F, OCHF₂, SCHF₂, and CH₂CN.

In some embodiments, R⁵ and R⁶ are independently selected from the group consisting of halide and C₁₋₃ alkyl.

In some embodiments, R⁵ and R⁶ together with the atom to which they are attached form a three to four membered carbocyclic ring optionally substituted with one or more halide, or a four membered heterocyclic ring. In some such embodiments, R⁵ and R⁶ together with the atom to which they are attached form a ring selected from the group consisting of cyclobutyl, 3-fluoro-cyclobutyl, 2-oxetanyl, 3-oxetanyl, and cyclopropyl

In some embodiments, A is selected from the group consisting of C—H, C-halide, and N. In some such embodiments, A is selected from the group consisting of C—H, C—Cl, and N.

In some embodiments, each X is independently selected from O or S.

In some embodiments, R¹ and R² are independently selected from the group consisting of H, —CH₃, CN, —CH₂OH, —CH₂F, —CHF₂ and F.

In some embodiments, R¹ and R² are linked to form a ring which selected from the group consisting of:

[chemical structure]

In some embodiments, R³ and R⁴ are independently selected from the group consisting of —CH₃, CN, —CH₂OH, —CH₂F, —CHF₂, —OCH₃, SCH₃, —OCH₂F, —SCH₂F, —OCHF₂, —SCHF₂, and F.

In some embodiments, R³ and R⁴ are linked to form a ring which selected from the group consisting of:

[chemical structure]

In some embodiments, R⁵ and R⁶ are independently selected from the group consisting of H, —CH₃, CN, —CH₂OH, —CH₂F, —CHF₂, —OCH₃, SCH₃, —OCH₂F, —SCH₂F, —OCHF₂, —SCHF₂, and F.

In some embodiments, R⁵ and R⁶ are linked to form a ring which selected from the group consisting of:

[chemical structure]

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### DEFINITIONS

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can be saturated or unsaturated (e.g., containing —C═C— or —C≡C— subunits), at one or several positions. Typically, alkyl groups will comprise 1 to 9 carbon atoms, preferably 1 to 6, more preferably 1 to 4, and most preferably 1 to 3 carbon atoms.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that at least one ring in the ring system is not aromatic. Typically, carbocyclyl groups will comprise 3 to 10 carbon atoms, preferably 3 to 4.

As used herein, “halo”, “halide” or “halogen” is a chloro, bromo, fluoro or iodo atom radical. Chloro, bromo and fluoro are preferred halides. Most preferred halide is fluorine.

As used herein, “haloalkyl” means a hydrocarbon substituent, which is a linear or branched alkyl, alkenyl or alkynyl substituted with chloro, bromo, fluoro or iodo atom(s). Most preferred of these are fluoroalkyls, wherein one or more of the hydrogen atoms have been substituted by fluoro. Preferred haloalkyls are of 1 to about 3 carbons in length, more preferred haloalkyls are 1 to about 2 carbons, and most preferred are 1 carbon in length. The skilled artisan will recognize then that as used herein, “haloalkylene” means a diradical variant of haloalkyl, such diradicals may act as spacers between radicals, other atoms, or between the parent ring and another functional group.

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “heterocyclyl” means a cyclic ring system comprising at least one heteroatom in the ring system backbone. Heterocyclyls may include multiple fused rings. Heterocyclyls may have any degree of saturation provided that at least one ring in the ring system is not aromatic. More preferred heterocycles are of 4-7 members. Most preferred heterocycles are 4 membered. In six membered monocyclic heterocycles, the heteroatom(s) are selected from one up to three of O, N or S, and wherein when the heterocycle is five membered, preferably it has one or two heteroatoms selected from O, N, or S. Examples of heterocyclyl include azirinyl, aziridinyl, azetidinyl, oxetanyl, thietanyl, 1,4,2-dithiazolyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, oxetanyl, and others.

When two R groups are said to form a ring (e.g., a carbocyclyl or heterocyclyl ring) “together with the atom to which they are attached,” it is meant that the collective unit of the atom and the two R groups are the recited ring. The ring is not otherwise limited by the definition of each R group when taken individually. For example, when the following substructure is present:

[chemical structure]

and R¹ and R² are defined as selected from the group consisting of hydrogen and alkyl, or R¹ and R² together with the nitrogen to which they are attached form a heterocycle, it is meant that R¹ and R² can be selected from hydrogen or alkyl, or alternatively, the substructure has structure:

[chemical structure]

where ring A is a heterocycle ring containing the depicted nitrogen.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “administration” or “administering” refers to a method of giving a dosage of a compound or pharmaceutical composition to a vertebrate or invertebrate, including a mammal, a bird, a fish, or an amphibian, where the method is, e.g., orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic device. The preferred method of administration can vary depending on various factors, e.g., the components of the pharmaceutical composition, the site of the disease, the disease involved, and the severity of the disease.

A “diagnostic” as used herein is a compound, method, system, or device that assists in the identification and characterization of a health or disease state. The diagnostic can be used in standard assays as is known in the art.

The term “mammal” is used in its usual biological sense. Thus, it specifically includes humans, cattle, horses, dogs, and cats, but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

Some embodiments include use of a therapeutically effective amount or a pharmaceutically effective amount of a compound disclosed herein. A “therapeutically effective amount” or “pharmaceutically effective amount” of a compound as provided herein is one which is sufficient to achieve the desired effect and may vary according to the nature and severity of the disease condition, and the potency of the compound. “Therapeutically effective amount” is also intended to include one or more of the compounds of Formula (I) in combination with one or more other agents that are effective to treat a microbial infection and/or conditions. The combination of compounds is preferably a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

A therapeutic effect relieves, to some extent, one or more of the symptoms of the disease, and includes curing a disease. “Curing” means that the symptoms of active disease are eliminated. However, certain long-term or permanent effects of the disease may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “therapeutic treatment” refers to administering treatment to a patient already suffering from a disease thus causing a therapeutically beneficial effect, such as ameliorating existing symptoms, preventing additional symptoms, ameliorating or preventing the underlying metabolic causes of symptoms, postponing or preventing the further development of a disorder and/or reducing the severity of symptoms that will or are expected to develop. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition.

### Administration and Pharmaceutical Compositions

Some embodiments include pharmaceutical compositions comprising: (a) a safe and therapeutically effective amount of a compound provided herein, or a pharmaceutically acceptable salt thereof; and (b) a pharmaceutically acceptable carrier, diluent, excipient or combination thereof.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, ontologically, neuro-otologically, intraocularly, subconjuctivally, via anterior eye chamber injection, intravitreally, intraperitoneally, intrathecally, intracystically, intrapleurally, via wound irrigation, intrabuccally, intra-abdominally, intra-articularly, intra-aurally, intrabronchially, intracapsularly, intrameningeally, via inhalation, via endotracheal or endobronchial instillation, via direct instillation into pulmonary cavities, intraspinally, intrasynovially, intrathoracically, via thoracostomy irrigation, epidurally, intratympanically, intracisternally, intravascularly, intraventricularly, intraosseously, via irrigation of infected bone, or via application as part of any admixture with a prosthetic devices. Oral and parenteral administrations are customary in treating the indications.

Compounds provided herein intended for pharmaceutical use may be administered as crystalline or amorphous products. Pharmaceutically acceptable compositions may include solid, semi-solid, liquid, solutions, colloidal, liposomes, emulsions, suspensions, complexes, coacervates and aerosols. Dosage forms, such as, e.g., tablets, capsules, powders, liquids, suspensions, suppositories, aerosols, implants, controlled release or the like. They may be obtained, for example, as solid plugs, powders, or films by methods such as precipitation, crystallization, milling, grinding, supercritical fluid processing, coacervation, complex coacervation, encapsulation, emulsification, complexation, freeze drying, spray drying, or evaporative drying. Microwave or radio frequency drying may be used for this purpose. The compounds can also be administered in sustained or controlled release dosage forms, including depot injections, osmotic pumps, pills (tablets and or capsules), transdermal (including electrotransport) patches, implants and the like, for prolonged and/or timed, pulsed administration at a predetermined rate.

The compounds can be administered either alone or more typically in combination with a conventional pharmaceutical carrier, excipient or the like. The term “excipient” is used herein to describe any ingredient other than the compound(s) provided herein. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-b-cyclodextrins, or other solubilized derivatives can also be advantageously used to enhance delivery of compounds described herein. Dosage forms or compositions containing a compound as described herein in the range of 0.005% to 100% with the balance made up from non-toxic carrier may be prepared. The contemplated compositions may contain 0.001%-100% active ingredient, in one embodiment 0.1-95%, in another embodiment 75-85%, in a further embodiment 20-80%, Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In one preferred embodiment, the compositions will take the form of a unit dosage form such as a pill or tablet and thus the composition may contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which the two active ingredients are physically separated are also contemplated; e.g., capsules with granules (or tablets in a capsule) of each drug; two-layer tablets; two-compartment gel caps, etc. Enteric coated or delayed release oral dosage forms are also contemplated.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrine derivatives, sorbitan monolaurate, triethanolamine acetate, triethanolamine oleate, and the like).

In some embodiments, the unit dosage of compounds of Formula (I) is 0.25 mg/Kg to 120 mg/Kg in humans.

In some embodiments, the unit dosage of compounds of Formula (I) is 0.50 mg/Kg to 70 mg/Kg in humans.

In some embodiments, the unit dosage of compounds of Formula (I) is 1.0 mg/Kg to 50 mg/Kg in humans.

In some embodiments, the unit dosage of compounds of Formula (I) is 1.50 mg/Kg to 10 mg/Kg in humans.

In some embodiments, the compositions are provided in unit dosage forms suitable for single administration of a precise dose.

In some embodiments, the compositions are provided in unit dosage forms suitable for twice a day administration of a precise dose.

In some embodiments, the compositions are provided in unit dosage forms suitable for three times a day administration of a precise dose.

Injectables can be prepared in conventional forms, either as liquid solutions, colloid, liposomes, complexes, coacervate or suspensions, as emulsions, or in solid forms suitable for reconstitution in liquid prior to injection. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The percentage of active compound contained in such parenteral compositions is highly dependent on the specific nature thereof, as well as the activity of the compound and the needs of the subject. However, percentages of active ingredient of 0.01% to 10% in solution are employable, and could be higher if the composition is a solid or suspension, which could be subsequently diluted to the above percentages.

In some embodiments, the composition will comprise 0.1-10% of the active agent in solution.

In some embodiments, the composition will comprise 0.1-5% of the active agent in solution.

In some embodiments, the composition will comprise 0.1-4% of the active agent in solution.

In some embodiments, the composition will comprise 0.15-3% of the active agent in solution.

In some embodiments, the composition will comprise 0.2-2% of the active agent in solution.

In some embodiments, the compositions are provided in dosage forms suitable for continuous dosage by intravenous infusion over a period of 1-96 hours.

In some embodiments, the compositions are provided in dosage forms suitable for continuous dosage by intravenous infusion over a period of 1-72 hours.

In some embodiments, the compositions are provided in dosage forms suitable for continuous dosage by intravenous infusion over a period of 1-48 hours.

In some embodiments, the compositions are provided in dosage forms suitable for continuous dosage by intravenous infusion over a period of 1-24 hours.

In some embodiments, the compositions are provided in dosage forms suitable for continuous dosage by intravenous infusion over a period of 1-12 hours.

In some embodiments, the compositions are provided in dosage forms suitable for continuous dosage by intravenous infusion over a period of 1-6 hours.

In some embodiments, these compositions can be administered by intravenous infusion to humans at doses of 5 mg/m² to 300 mg/m².

In some embodiments, these compositions can be administered by intravenous infusion to humans at doses of 5 mg/m² to 200 mg/m².

In some embodiments, these compositions can be administered by intravenous infusion to humans at doses of 5 mg/m² to 100 mg/m².

In some embodiments, these compositions can be administered by intravenous infusion to humans at doses of 10 mg/m² to 50 mg/m².

In some embodiments, these compositions can be administered by intravenous infusion to humans at doses of 50 mg/m² to 200 mg/m².

In some embodiments, these compositions can be administered by intravenous infusion to humans at doses of 75 mg/m² to 175 mg/m².

In some embodiments, these compositions can be administered by intravenous infusion to humans at doses of 100 mg/m² to 150 mg/m².

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In one preferred embodiment, the compositions can be administered to the respiratory tract (including nasal and pulmonary) e.g., through a nebulizer, metered-dose inhalers, atomizer, mister, aerosol, dry powder inhaler, insufflator, liquid instillation or other suitable device or technique.

In some embodiments, aerosols intended for delivery to the nasal mucosa are provided for inhalation through the nose. For optimal delivery to the nasal cavities, inhaled particle sizes of about 5 to about 100 microns are useful, with particle sizes of about 10 to about 60 microns being preferred. For nasal delivery, a larger inhaled particle size is desired to maximize impaction on the nasal mucosa and to minimize or prevent pulmonary deposition of the administered formulation. In some embodiments, aerosols intended for delivery to the lung are provided for inhalation through the nose or the mouth. For optimal delivery to the lung, inhaled aerodynamic particle sizes of about less than 10 μm are useful, with an aerodynamic particle size of about 1 to about 10 microns being preferred. Inhaled particles may be defined as liquid droplets containing dissolved drug, liquid droplets containing suspended drug particles (in cases where the drug is insoluble in the suspending medium), dry particles of pure drug substance, drug substance incorporated with excipients, liposomes, emulsions, colloidal systems, coacervates, aggregates of drug nanoparticles, or dry particles of a diluent which contain embedded drug nanoparticles.

In some embodiments, compounds of Formula (I) disclosed herein intended for respiratory delivery (either systemic or local) can be administered as aqueous formulations, as non-aqueous solutions or suspensions, as suspensions or solutions in halogenated hydrocarbon propellants with or without alcohol, as a colloidal system, as emulsions, coacervates or as dry powders. Aqueous formulations may be aerosolized by liquid nebulizers employing either hydraulic or ultrasonic atomization or by modified micropump systems (like the soft mist inhalers, the Aerodose or the AERx® systems). Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

In some embodiments, the compositions of Formula (I) disclosed herein can be administered to the ear by various methods. For example, a round window catheter (e.g., U.S. Pat. Nos. 6,440,102 and 6,648,873) can be used.

Alternatively, formulations can be incorporated into a wick for use between the outer and middle ear (e.g., U.S. Pat. No. 6,120,484) or absorbed to collagen sponge or other solid support (e.g., U.S. Pat. No. 4,164,559).

If desired, formulations of the invention can be incorporated into a gel formulation (e.g., U.S. Pat. Nos. 4,474,752 and 6,911,211).

In some embodiments, compounds of Formula (I) disclosed herein intended for delivery to the ear can be administered via an implanted pump and delivery system through a needle directly into the middle or inner ear (cochlea) or through a cochlear implant stylet electrode channel or alternative prepared drug delivery channel such as but not limited to a needle through temporal bone into the cochlea.

Other options include delivery via a pump through a thin film coated onto a multichannel electrode or electrode with a specially imbedded drug delivery channel (pathways) carved into the thin film for this purpose. In other embodiments the acidic or basic solid gacyclidine can be delivered from the reservoir of an external or internal implanted pumping system.

Formulations of the invention also can be administered to the ear by intratympanic injection into the middle ear, inner ear, or cochlea (e.g., U.S. Pat. No. 6,377,849 and Ser. No. 11/337,815).

Intratympanic injection of therapeutic agents is the technique of injecting a therapeutic agent behind the tympanic membrane into the middle and/or inner ear. In one embodiment, the formulations described herein are administered directly onto the round window membrane via transtympanic injection. In another embodiment, the ion channel modulating agent auris-acceptable formulations described herein are administered onto the round window membrane via a non-transtympanic approach to the inner ear. In additional embodiments, the formulation described herein is administered onto the round window membrane via a surgical approach to the round window membrane comprising modification of the crista fenestrae cochleae.

In some embodiments, the compounds of Formula (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG (like PEG ointments), and the like.

Suppositories for rectal administration of the drug (either as a solution, colloid, suspension or a complex) can be prepared by mixing the drug with a suitable non-irritating excipient that is solid at ordinary temperatures but liquid at the rectal temperature and will therefore melt or erode/dissolve in the rectum and release the drug. Such materials include cocoa butter, glycerinated gelatin, hydrogenated vegetable oils, poloxamers, mixtures of polyethylene glycols of various molecular weights and fatty acid esters of polyethylene glycol. In suppository forms of the compositions, a low-melting wax such as, but not limited to, a mixture of fatty acid glycerides, optionally in combination with cocoa butter is first melted.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

Solid compositions can be provided in various different types of dosage forms, depending on the physicochemical properties of the drug, the desired dissolution rate, cost considerations, and other criteria. In one of the embodiments, the solid composition is a single unit. This implies that one unit dose of the drug is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In a preferred embodiment, the solid composition is a highly porous lyophilized form. Such lyophilizates, sometimes also called wafers or lyophilized tablets, are particularly useful for their rapid disintegration, which also enables the rapid dissolution of the active compound.

On the other hand, for some applications the solid composition may also be formed as a multiple unit dosage form as defined above. Examples of multiple units are powders, granules, microparticles, pellets, mini-tablets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution. Effervescent compositions are also contemplated to aid the quick dispersion and absorption of the compound.

Another type of multiparticulate system which is also capable of achieving rapid drug dissolution is that of powders, granules, or pellets from water-soluble excipients which are coated with the drug, so that the drug is located at the outer surface of the individual particles. In this type of system, the water-soluble low molecular weight excipient is useful for preparing the cores of such coated particles, which can be subsequently coated with a coating composition comprising the drug and, preferably, one or more additional excipients, such as a binder, a pore former, a saccharide, a sugar alcohol, a film-forming polymer, a plasticizer, or other excipients used in pharmaceutical coating compositions.

### Methods of Treatment

Some embodiments of the present invention include methods of treating bacterial infections with the compounds and compositions comprising compounds described herein. Some methods include administering a compound, composition, pharmaceutical composition described herein to a subject in need thereof. In some embodiments, a subject can be an animal, e.g., a mammal, a human. In some embodiments, the bacterial infection comprises a bacteria described herein. As will be appreciated from the foregoing, methods of treating a bacterial infection include methods for preventing bacterial infection in a subject at risk thereof.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Examples of additional medicaments include an antibacterial agent, antifungal agent, an antiviral agent, an anti-inflammatory agent and an anti-allergic agent.

Some embodiments include a combination of the compounds, compositions and/or pharmaceutical compositions described herein with an additional agent, wherein the additional agent comprises a β-lactamase inhibitor. Non-limiting examples of β-lactamase inhibitors include clavulanic acid, tazobactam, sulbactam, RPX-7009, NXL104, MK-7655, BAL-29880, SYN-2190, BLI-489, AM-112, and ME1071. In various embodiments, the β-lactamase inhibitor is a class A, B, C, or D β-lactamase inhibitor. An example of a class B β-lactamase inhibitor includes ME1071 [Antimicrob. Agents Chemother. (2010), 54(9), 3625-3629]. Some embodiments include co-administering the compound, composition or pharmaceutical composition described herein with one or more additional agents.

### Indications

The compounds and compositions comprising monocyclic β-lactam derivatives described herein can be used to treat bacterial infections. Bacterial infections that can be treated with the compounds, compositions and methods described herein can comprise a wide spectrum of bacteria.

Examples of bacterial infections include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### EXAMPLES

### Compound Preparation

The starting materials used in preparing the compounds of the invention are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protective Groups in Organic Synthesis, 4th Ed., John Wiley & Sons (2007).

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

(^{C}H) nuclear magnetic resonance spectra (NMR) were measured in the indicated solvents on a Bruker NMR spectrometer (Avance TM DRX300, 300 MHz for ¹H or Avance TM DRX500, 500 MHz for ¹H) or Varian NMR spectrometer (Mercury 400BB, 400 MHz for ¹H). Peak positions are expressed in parts per million (ppm) downfield from tetramethylsilane. The peak multiplicities are denoted as follows, s, singlet; d, doublet; t, triplet; q, quartet; ABq, AB quartet; quin, quintet; sex, sextet; sep, septet; non, nonet; dd, doublet of doublets; d/ABq, doublet of AB quartet; dt, doublet of triplets; td, triplet of doublets; dq, doublet of quartets; m, multiplet.

The following abbreviations have the indicated meanings:

brine=saturated aqueous sodium chloride

CDCl₃=deuterated chloroform

DCC=N,N′-dicyclohexylcarbodiimide

DCM=dichloromethane

DEAD=diethyl azodicarboxylate

DMF=N,N-dimethylformamide

DMSO-d₆=deuterated dimethylsulfoxide

ESIMS=electron spray mass spectrometry

EtOAc=ethyl acetate

HCl=hydrochloric acid

K₂CO₃=potassium carbonate

LC/MS=liquid chromatography-mass spectrometry

MeOH=methanol

MTBE=methyl tert-butyl ether

NMR=nuclear magnetic resonance

PCl₅=phosphorus pentachloride

Pd/C=palladium on carbon

PPh₃=triphenylphosphine

rt=room temperature

TFA=trifluoroacetic acid

THF=tetrahydrofuran

TLC=thin layer chromatography

The following example schemes are provided for the guidance of the reader, and collectively represent an example method for making the compounds provided herein. Furthermore, other methods for preparing compounds of the invention will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application. Unless otherwise indicated, all variables are as defined above.

### General Procedures

Intermediates for the β-lactam core fragment are synthesized as shown in general Schemes 1-4. Preparations of requisite β-hydroxy-α-aminoacids can be achieved by some of the methods described in the literature [Tetrahedron Letters (1986), 27(25), 2793-2796; Journal of Biological Chemistry (1949), 178(2), 709-714; Aktiengesellschaft, D., Process for the production of beta-hydroxy-alpha-aminocarboxylic acids, A. Kleemann, B. Lehmann, K. Deller—U.S. Pat. No. 4,486,600, Dec. 4, 1984]. When necessary desired single stereoisomers can be obtained by resolution of salts with optically pure amines or acids as applicable [Slusarchyk, William A.; Koster, William H., [3 S(Z)]-2-[[[1-(2-amino-4-thiazolyl)-2-[[2,2-dimethyl-4-oxo-1-(sulfooxy)-3-azetidinyl]amino]-2-oxoethylidene]-amino]oxy]acetic acid and intermediate, U.S. Pat. No. 4,638,061, Jan. 20, 1987.; The Journal of Peptide Research (1998), 52(2), 143-15]. Alternatively optically pure starting materials or chiral catalysts may be employed in stereoselective synthetic sequences leading to single stereoisomers [Tetrahedron (1983), 39(12), 2085-2092; Tetrahedron (1988), 44(17), 5277-5292; Journal of the American Oil Chemists' Society (1997), 74(11), 1345-1360; Tetrahedron: Asymmetry (2001), 12(3), 481-486; The Journal of Organic Chemistry (2002), 68(1), 177-179].

In following schemes, P₁-P₇ denotes typical protecting groups compatible with requisite chemistry and L denotes typical leaving groups. In cases when unprotected intermediates are suitable for requisite transformations P₁-P₇ may also denote hydrogen.

Preparation of the β-lactam core intermediates X and XI from bis N-protected glycine ester (II) or mono N-protected glycine ester (V) is shown in Scheme 1.

[chemical structure]

Scheme 1 describes a method for preparation of the β-lactam core intermediates X and XI by either starting with bis N-protected glycine ester (II) [Tetrahedron (1988), 44(12), 3685-3692] which is first reacted with ketone (III) followed by bis deprotection of the amine and then monoprotection to give β-hydroxyacid (VI). β-hydroxyacid (VI) can also be synthesized starting with the mono N-protected glycine ester (II) [Tetrahedron Letters (1986), 27(25), 2789-2792; Sundeen, J. E. (1987, July). Preparation of monosulfactams as antibiotics. Eur. Pat. Appl. EP0229012, Squibb and Sons, Inc., USA; Sundeen, J. E. (1987, July). O-Sulfated spiro β-lactam hydroxamic acids. U.S. Pat. No. 4,680,388, Squibb and Sons, Inc., USA] through a similar path without the need to deprotect/protect the amine. β-hydroxyacid (VI) is then coupled with a protected hydroxylamine (VII) followed by either cyclization by the Mitsunobu reaction or 2-picoline (or pyridine)-SO₃ complex to give the protected β-lactam (IX). IX can be first N-deprotected to give intermediate X or further O-deprotected to give intermediate XI.

Another preparation of the β-lactam core intermediates X and XI from copper glycinate (XII) is shown in Scheme 2.

[chemical structure]

Scheme 2 describes a method for preparation of the β-lactam core intermediates X and XI which starts with the alkylation [Hoppe-Seyler's Zenschrift fuer Physiologische Chemie (1961), 327, 41-48] of a copper(II)glycinate derivative (XII) with ketone (III) to yield a β-hydroxyacid (IV). β-hydroxyacid (VI) is N-protected before then being coupled with a protected hydroxylamine (VII) followed by either cyclization by the Mitsunobu reaction or 2-picoline (or pyridine)-SO₃ complex to give the protected β-lactam (XIII) XIII can be first N-deprotected to give intermediate XIV or further O-deprotected to give intermediate XV. All three racemic β-lactams can be optically resolved by known procedures [Slusarchyk, William A.; Koster, William H., [3S(Z)]-2-[[[1-(2-amino-4-thiazolyl)-2-[[2,2-dimethyl-4-oxo-1-(sulfooxy)-3-azetidinyl]amino]-2-oxoethylidene]-amino]oxy]acetic acid and intermediate, U.S. Pat. No. 4,638,061, Jan. 20, 1987.; The Journal of Peptide Research (1998), 52(2), 143-15].

Preparation of the β-lactam core intermediate XIX from mono N-protected glycine ester (V) and an aminoxyacetic acid derivative (XVI) is shown in Scheme 3.

[chemical structure]

Scheme 3 describes a method [Tetrahedron Letters (1986), 27(25), 2789-2792; Sundeen, J. E. (1987, July). Preparation of monosulfactams as antibiotics. Eur. Pat. Appl. EP0229012, Squibb and Sons, Inc., USA; Sundeen, J. E. (1987, July). O-Sulfated spiro β-lactam hydroxamic acids. U.S. Pat. No. 4,680,388, Squibb and Sons, Inc., USA] for preparation of the β-lactam core intermediate XIX which starts by coupling a N-protected β-hydroxyacid (VI) with a aminoxyacetic acid derivative (XVI) to produce XVII which is cyclized either by the Mitsunobu reaction or 2-picoline (or pyridine)-SO₃ complex to give the protected β-lactam (XVIII). XVIII is then N-deprotected to give intermediate XIX.

Another preparation of the β-lactam core intermediate XIX from mono N-protected glycine ester (XX) and an α-haloacetic acid derivative (XXI) is shown in Scheme 4.

[chemical structure]

Another preparation of the β-lactam core intermediate XIX is shown above in Scheme 4 with protected β-lactam IX being first O-deprotected followed by coupling with an α-haloacetic acid derivative (XXI) to produce XVIII XVIII is then N-deprotected to give intermediate XIX.

General schemes 5-6 describe synthetic routes by which monocyclic β-lactam intermediates are coupled to N-acyl sidechain and later converted to final monocyclic β-lactams of current invention. The following schemes assume proper selection of orthogonal protecting groups allowing selective unmasking of requisite functionalities.

Compounds of Formula I of the present invention can be prepared as depicted in Scheme 5.

[chemical structure]

[chemical structure]

Scheme 5 describes a method [European Journal of Medicinal Chemistry (1981), 16(4), 307-316] for preparation of monobactam derivatives (XXXII) from the protected carboxymethoxyiminoacetic acid intermediate XXVIII XXVIII can be synthesized by coupling hydroxyimino derivative (XXII or XXIII) with the protected acetic acid derivative (XXIV) followed any necessary deprotection or by direct coupling of the aminoxyacetic acid derivative (XXVII) with glycolic acid derivative (XXVI). XXVIII can then be coupled with O-protected β-lactam derivative (X) or with unprotected β-lactam derivative (XI) to ultimately give monobactam derivative (XXX). The free hydroxy of XXX is then coupled with protected α-haloacetic acid derivative (XXI) to yield fully protected monobactam derivative (XXXI) which can be deprotected to give the desired final monobactam derivatives (XXXII).

Compounds of Formula I of the present invention can alternatively be prepared as depicted in Scheme 6.

[chemical structure]

Scheme 6 describes an alternative method for preparation of monobactam derivatives (XXXII) by coupling 3-amino-β-lactam (XIX) with the protected carboxymethoxyiminoacetic acid intermediate XXVIII to give fully protected monobactam derivative (XXXI) which can be deprotected to give the desired final monobactam derivatives (XXXII).

### ILLUSTRATIVE COMPOUND EXAMPLES

Preparation of intermediate (XXXVI) is depicted below in Scheme 7.

[chemical structure]

### Step 1-2

To a suspension of sodium hydride (2.0 g, 60% dispersion in mineral oil, prewashed with hexane) in anhydrous DMF (150 mL) at 0° C. was added phtalimide (XXXIII) (8.15 g, 50.0 mmol) in portions. After complete addition, stirring continued for 30 min at room temperature before adding tert-butyl 2-bromoacetate (XXXIV) (10.7 g [8 mL], 54.9 mmol). The reaction was stirred for 30 min, and then the solution was poured on 100 g ice. The precipitate was filtered and air dried. The crude material was dissolved in DCM/MeOH (75:10) mixture followed by a 97% solution of hydrazine hydrate (6.3 mL [64-65% hydrazine], 117 mmol) added at room temperature (some exothermic heating was observed). The precipitate was filtered and the filtrate was concentrated under reduced pressure to yield tert-butyl 2-(aminooxy)acetate (XXXVI) as clear liquid. (6.9 g, 46.9 mmol, 93.8% yield). ESIMS found C₆H₁₃NO₃ m/z 148 (M+H).

Preparation of intermediate (XLII) is depicted below in Scheme 8.

[chemical structure]

### Step 1

To a solution of (S)-2-(tert-butoxycarbonylamino)-3-hydroxy-3-methylbutanoic acid (XXXVII) (851 mg, 3.65 mmol) in anhydrous THF (20 mL) was added HOBT (493 mg, 3.65 mmol). The reaction mixture was cooled in a salt/ice bath to −10° C. which was followed by addition of DCC (752 mg, 3.65 mmol). The reaction mixture was stirred at 0° C. for 40 min before adding a solution of O-benzylhydroxylamine (XXXVIII) (538 mg, 4.37 mmol) in THF (5 mL). Stirring continued 0° C. followed by warming to room temperature for 1.5 h. To the reaction mixture was added hexane (20 mL), the precipitate was filtered and the filtrate was concentrated under vacuum. To the oily residue was chromatographed on silica gel (1:1 EtOAc/hexane) followed by recrystallization from MTBE to produce (S)-tert-butyl 1-(benzyloxyamino)-3-hydroxy-3-methyl-1-oxobutan-2-ylcarbamate (XXXIX) (1.002 g, 2.96 mmol, 81.1% yield). ESIMS found C₁₇H₂₆N₂O₅ m/z 339 (M+H).

### Step 2

A solution of (S)-tert-butyl 1-(benzyloxyamino)-3-hydroxy-3-methyl-1-oxobutan-2-ylcarbamate (XXXIX) (950 mg, 2.81 mmol) and 2-picoline-SO₃ complex (630 mg, 3.64 mmol, freshly prepared) in 10 mL of 2-picoline was stirred at room temperature for few hours. The solvent was evaporated and the residue was refluxed with EtOAc (10 mL) and aqueous solution of potassium carbonate (prepared by dissolving 2.3 g K₂CO₃ in 5 mL water) for few hours. The organic solution was washed with water, 1 M citric acid solution and dried over anhydrous sodium sulfate. After evaporation the crude material was purified on a silica gel cartridge (2:1 hexane/EtOAc) to give (S)-tert-butyl 1-(benzyloxy)-2,2-dimethyl-4-oxoazetidin-3-ylcarbamate (XL) as a crystalline solid (380 mg, 1.19 mmol, 42.2% yield). ESIMS found C₁₇H₂₄N₂O₄ m/z 321 (M+H).

### Step 3

To a solution of (S)-tert-butyl 1-(benzyloxy)-2,2-dimethyl-4-oxoazetidin-3-ylcarbamate (XL) (450 mg, 1.40 mmol) in EtOH (10 mL) was added 10% Pd/C (50 mg). The mixture was stirred vigorously under atmospheric pressure of hydrogen. After 2 h, the catalyst was filtered and solvent was removed under vacuum to yield (S)-tert-butyl 1-hydroxy-2,2-dimethyl-4-oxoazetidin-3-ylcarbamate (XLI) (340 mg, 1.48 mmol, quantitative yield). ESIMS found C₁₀H₁₈N₂O₄ m/z 231 (M+H).

### Step 4

To a solution (S)-tert-butyl 1-hydroxy-2,2-dimethyl-4-oxoazetidin-3-ylcarbamate (XLI) (108 mg, 0.47 mmol) in DCM (0.5 mL) cooled in an ice-water bath to 0° C. was added triethylsilane (50 mg, 43.0 mmol) followed by TFA (0.5 mL). The reaction was stirred for 30 min and concentrated under vacuum. DCM was added and reaction mixture was evaporated to dryness producing crude (S)-3-amino-1-hydroxy-4,4-dimethylazetidin-2-one (XLII) which was used in Example 1, Step 3 without further purification. ESIMS found C₅H₁₀N₂O₂ m/z 131 (M+H).

### Example 1

Preparation of (S,Z)-2-(1-(2-aminothiazol-4-yl)-2-(1-(carboxymethoxy)-2,2-dimethyl-4-oxoazetidin-3-ylamino)-2-oxo ethylideneaminooxy) acetic acid (1) is depicted below in Scheme 14.

[chemical structure]

### Step 1

To a solution of ethyl 2-oxo-2-(2-(tritylamino)thiazol-4-yl)acetate (XLIII) (8.3 g, 18.8 mmol) in MeOH (8 mL) was added a solution of methanolic NaOH (19.5 mL, 1.0 M). The solution was refluxed for 10 min (LC/MS shows no ester present), cooled and filtered. The solid was then suspended in water followed by addition of 1M HCl (17 mL). The yellow precipitate was filtered, dissolved in 1,4-dioxane and evaporated under vacuum to give 2-oxo-2-(2-(tritylamino)thiazol-4-yl)acetic acid (XLIV) (7.8 g, 18.8 mmol, 100% yield). ESIMS found C₂₄H₁₈N₂O₃S m/z 415 (M+H).

### Step 2

To a −10° C. solution of tert-butyl 2-(aminooxy)acetate (XXXVI) (0.75 g, 5.1 mmol) in DCM (10 mL) was slowly added a solution of 2-oxo-2-(2-(tritylamino)thiazol-4-yl)acetic acid (XLIV) (2.13 g, 5.1 mmol) in DCM (10 mL). The reaction was stored in a −20° C. freezer for 3 days. The precipitate was filtered and dried to produce (Z)-2-(2-tert-butoxy-2-oxoethoxyimino)-2-(2-(tritylamino)thiazol-4-yl)acetic acid (XLV) as a solid. (2.1 g, 3.9 mmol, 76.5% yield). ESIMS found C₃₀H₂₉N₃O₅S m/z 544 (M+H).

### Step 3

A solution of (Z)-2-(2-tert-butoxy-2-oxoethoxyimino)-2-(2-(tritylamino)thiazol-4-yl)acetic acid (XLV) (0.55 g, 1.0 mmol) in DCM (3 mL) and cooled in ice bath was added a solution of PCl₅ in DCM (3.5 mL, 0.35 M) over a 5 min period. After 30 min, an additional volume of the above solution PCl₅ in DCM (1.8 mL, 0.18 M) was added and the reaction was continued for another 15 min at 0° C. To the reaction mixture was added MTBE and the precipitate formed was filtered to yield crude (Z)-tert-butyl 2-(2-chloro-2-oxo-1-(2-(tritylamino)thiazol-4-yl)ethylideneaminooxy) acetate (0.55 g, 0.98 mmol, 97.9% yield) which was used in Step 4 without further purification.

### Step 4

To a solution of (S)-3-amino-1-hydroxy-4,4-dimethylazetidin-2-one (XLII) (76 mg, 0.58 mmol) in THF (1 mL) cooled in an ice bath was added triethylamine (67 mg, 0.66 mmol), followed by crude (Z)-tert-butyl 2-(2-chloro-2-oxo-1-(2-(tritylamino)thiazol-4-yl)ethylideneaminooxy)acetate (130 mg, 0.23) in THF (1.5 mL). The reaction was stirred for 15 min before the solution was partitioned between EtOAc and 1 M citric acid. The organic phase was washed with water, dried over sodium sulfate and evaporated. The crude oil (190 mg) was purified by silica gel chromatography (3:1 EtOAc/hexane) to give (S,Z)-tert-butyl 2-(2-(1-hydroxy-2,2-dimethyl-4-oxoazetidin-3-ylamino)-2-oxo-1-(2-(tritylamino)thiazol-4-yl)ethylideneaminooxy)acetate (XLVI). (50 mg, 0.077 mmol, 33.2% yield). ESIMS found C₃₅H₃₇N₅O₆S m/z 656 (M+H).

### Step 5

To a solution of (S,Z)-tert-butyl 2-(2-(1-hydroxy-2,2-dimethyl-4-oxoazetidin-3-ylamino)-2-oxo-1-(2-(tritylamino)thiazol-4-yl)ethylideneaminooxy)acetate (XLVI) (100 mg, 0.152 mmol) in DMF (1 mL) was added potassium carbonate (23 mg, 0.17) followed by tert-butylbromoacetate (XLVII) (30 mg, 0.077 mmol) The reaction was stirred for 1.5 h at room temperature before an additional portion of tert-butylbromoacetate (XLVII) (15 mg, 0.038 mmol) was added and stirring was continued for 30 min. The reaction was partitioned between EtOAc/hexane/water and the organic phase was washed with water and dried over sodium sulfate. The residue was purified on silica gel (hexane/EtOAc) to produce (S,Z)-tert-butyl 2-(2-(1-(2-tert-butoxy-2-oxoethoxy)-2,2-dimethyl-4-oxoazetidin-3-ylamino)-2-oxo-1-(2-(tritylamino)thiazol-4-yl)ethylideneaminooxy)acetate (XLVIII) (93 mg, 0.121 mmol, 79.5% yield). ESIMS found C₄₁H₄₇N₅O₈S m/z 770 (M+H).

### Step 6

To a solution of (S,Z)-tert-butyl 2-(2-(1-(2-tert-butoxy-2-oxoethoxy)-2,2-dimethyl-4-oxoazetidin-3-ylamino)-2-oxo-1-(2-(tritylamino)thiazol-4-yl) ethylideneaminooxy)acetate (XLVIII) (93 mg, 0.121 mmol) in ethylene chloride (2 mL) was added a mixture of ethylene chloride/TFA (4 mL) while cooling in an ice bath. The reaction was warmed to room temperature and stirred until no partial deprotection products can be observed by LCMS. Reaction mixture was concentrated under vacuum before diisopropylether was added. The precipitate was filtered yielding the crude product. The crude material was dissolved in water (1 mL) and filtered. The filtrate was freeze-dried to produce (S,Z)-2-(1-(2-aminothiazol-4-yl)-2-(1-(carboxymethoxy)-2,2-dimethyl-4-oxoazetidin-3-ylamino)-2-oxoethylideneaminooxy)acetic acid (1) as the trifluoroacetate salt (59 mg, 0.111 mmol, 92.1% yield). ¹H NMR (D₂O/TFA-d, 500 MHz) δ ppm 1.13 (s, 3H), 1.33 (s, 3H), 4.42 (s, 2H), 4.54 (s, 1H), 4.61 (s, 2H), 6.90 (s, 1H); ESIMS found C₄₁H₄₇N₅O₈S m/z 437.9 (M+Na)⁺.

Illustrative compounds of Formula (I) are shown in Table 1.

TABLE 1

| | |
|---|---|
| 1 | [chemical structure] |
| 2 | [chemical structure] |
| 3 | [chemical structure] |
| 4 | [chemical structure] |
| 5 | [chemical structure] |
| 6 | [chemical structure] |
| 7 | [chemical structure] |
| 8 | [chemical structure] |
| 9 | [chemical structure] |
| 10 | [chemical structure] |
| 11 | [chemical structure] |
| 12 | [chemical structure] |
| 13 | [chemical structure] |
| 14 | [chemical structure] |
| 15 | [chemical structure] |
| 16 | [chemical structure] |
| 17 | [chemical structure] |
| 18 | [chemical structure] |
| 19 | [chemical structure] |
| 20 | [chemical structure] |
| 21 | [chemical structure] |
| 22 | [chemical structure] |
| 23 | [chemical structure] |
| 24 | [chemical structure] |
| 25 | [chemical structure] |
| 26 | [chemical structure] |
| 27 | [chemical structure] |
| 28 | [chemical structure] |
| 29 | [chemical structure] |
| 30 | [chemical structure] |
| 31 | [chemical structure] |
| 32 | [chemical structure] |
| 33 | [chemical structure] |
| 34 | [chemical structure] |
| 35 | [chemical structure] |
| 36 | [chemical structure] |
| 37 | [chemical structure] |
| 38 | [chemical structure] |
| 39 | [chemical structure] |

### Example 2

Potency and spectrum of compound 1 was determined using the panel of isogenic engineered strains, each containing a plasmid carrying a cloned gene expressing a β-lactamase enzyme. The strain that contained the empty vector was included to evaluate the impact of individual β-lactamases on microbiological activity of compound 1 vs comparator antibiotics. The host strain was wild-type strain of Escherichia coli, ECM5497. The vector plasmid was pUCP24. Microbiological activity is defined as Minimal inhibitory concentration (MIC), or the lowest concentration of antibiotics, at which the visible growth of the organism is completely inhibited. MIC was assessed using broth microdilution method as recommended by the NCCLS (National Committee for Clinical Laboratory Standards. Methods for Dilution Antimicrobial Susceptibility Tests for Bacteria that Grow Aerobically—4^{th} Edition. Approved Standard NCCLS Document M7-A4, Vol. 17, No. 2, NCCLS, Wayne, Pa., January 1997). In this assay multiple dilutions of drugs are being tested for the ability to inhibit growth of the test bacterial strains. The MIC assay is performed in microtiter plates. Antibiotics are diluted two-fold in the x axis, each column containing a single concentration of antibiotic. The assay is performed in MHB with a final bacterial inoculum of 5×10⁵ CFU/mL (from an early-log phase culture). Microtiter plates are incubated during 20 h at 35° C. and are read using a microtiterplate reader (Molecular Devices) at 650 nm as well as visual observation using a microtiter plate reading mirror. The MIC is defined as the lowest concentration of antibiotics at which the visible growth of the organism is completely inhibited. Results are presented in Tables 2 and 3.

Table 2. Activity of Compound 1 and comparator antibiotics against E. coli strains expressing individual β-lactamases.

TABLE 2

| | | β-lactamase | MIC (mg/L) | | | | |
|---|---|---|---|---|---|---|---|
| Strain | Plasmid | class | Compound 1 | AZTREONAM | TIGEMONAM | CARUMONAM | CEFTAZIDIME |
| ECM6704 | pUCP24 | vector | 2 | 0.125 | 0.25 | ≦0.06 | 0.125 |
| ECM6694 | pUCP24::CTX-M-15 | A, ESBL | 1 | 32 | 2 | 0.125 | 16 |
| ECM6718 | pUCP24::SHV-5 | A, ESBL | 4 | 16 | 32 | 2 | 8 |
| ECM6713 | pUCP24::TEM-10 | A, ESBL | 16 | >64 | >64 | 4 | 65 |
| ECM6701 | pUCP24::KPC-2 | A, carbapenemase | 1 | 16 | 0.5 | 0.25 | 4 |
| ECM6715 | pUCP24::AmpC | C | 2 | 8 | 4 | 4 | 16 |
| ECM6716 | pUCP24::OXA-48 | D | 2 | 0.125 | 0.25 | ≦0.06 | 0.125 |
| ECM6703 | pUCP24::NDM-1 | B | 1 | ≦0.06 | 0.25 | ≦0.06 | >64 |
| ECM6711 | pUCP24::VIM-1 | B | 1 | ≦0.06 | 0.25 | ≦0.06 | >64 |
| ECM6706 | pUCP24::SME-2 | A, carbapenemase | 1 | >64 | 0.5 | 0.125 | 1 |
| ECM6696 | pUCP24::NMC-A | A, carbapenemase | 1 | 64 | 0.5 | 0.25 | 0.5 |

Table 3. Ratios of MIC of each antibiotic vs vector alone

TABLE 3

| | | β-lactamase | MIC (mg/L) | | | | |
|---|---|---|---|---|---|---|---|
| Strain | Plasmid | class | Compound 1 | AZTREONAM | TIGEMONAM | CARUMONAM | CEFTAZIDIME |
| ECM6704 | pUCP24 | vector | 1 | 1 | 1 | 1 | 1 |
| ECM6694 | pUCP24::CTX-M-15 | A, ESBL | 0.5 | 256 | 8 | 2 | 128 |
| ECM6718 | pUCP24::SHV-5 | A, ESBL | 2 | 128 | 128 | 32 | 64 |
| ECM6713 | pUCP24::TEM-10 | A, ESBL | 8 | 512 | 256 | 64 | 512 |
| ECM6701 | pUCP24::KPC-2 | A, carbapenemase | 0.5 | 128 | 2 | 5 | 32 |
| ECM6715 | pUCP24::AmpC | C | 1 | 64 | 16 | 64 | 128 |
| ECM6716 | pUCP24::OXA-48 | D | 1 | 1 | 2 | 1 | 1 |
| ECM6703 | pUCP24::NDM-1 | B | 0.5 | 0.5 | 2 | 1 | 512 |
| ECM6711 | pUCP24::VIM-1 | B | 0.5 | 0.5 | 1 | 1 | 512 |
| ECM6706 | pUCP24::SME-2 | A, carbapenemase | 0.5 | 512 | 2 | 2 | 8 |
| ECM6696 | pUCP24::NMC-A | A, carbapenemase | 0.5 | 512 | 2 | 4 | 4 |

Table 2 contains MICs of compound 1 and comparator antibiotics for each test strain and Table 3 contains MIC ratios vs vector only control (strain ECM6704). β-Lactamases present in the test panel belonged to all four currently described classes of enzymes, A, B, C and D. These β-lactamases also represent the major groups of the most relevant enzymes.

The results indicate that out of several β-lactams tested (monobactams aztreonam, tigemonam and carumonam and cephalosporin ceftazidime), compound 1 was the least affected by β-lactamases. Specifically, other than TEM-10, no enzyme in this panel affected the potency of compound 1. Similar to other monobactams, it was not affected by class B or class D enzymes. But unlike other monobactams, it was not affected by the majority of class A and class C enzymes as well. The only enzyme in this panel that had some effect on activity of compound 1 was TEM-10. Still even the effect of TEM-10 on compound 1 was still much less than that on other monobactams or ceftazidime.

The term “comprising” as used herein is synonymous with “including,” “containing,” or “characterized by,” and is inclusive or open-ended and does not exclude additional, unrecited elements or method steps.

What is claimed is:

1. A compound having the structure of formula I: [chemical structure]
or a pharmaceutically acceptable salt thereof, wherein:
R¹ and R² are independently selected from the group consisting of H, halide, CN, C₁₋₃ alkyl, —X(C₁₋₃ alkyl), —(C₁₋₃ alkyl)OH, —(C₁₋₃ alkyl)CN and C₁₋₃ haloalkyl, or alternatively R¹ and R² together with the atom to which they are attached form a three to four membered carbocyclic ring optionally substituted with one or more halide, or a four membered heterocyclic ring;
R³ and R⁴ are independently selected from the group consisting of halide, CN, C₁₋₃ alkyl, —X(C₁₋₃ alkyl), —X(C₁₋₃ haloalkyl), —(C₁₋₃ alkyl)OH, —(C₁₋₃ alkyl)CN and C₁₋₃ haloalkyl, or alternatively R³ and R⁴ together with the atom to which they are attached form a three to four membered carbocyclic ring optionally substituted with one or more halide, or a four membered heterocyclic ring;
R⁵ and R⁶ are independently selected from the group consisting of H, halide, CN, C₁₋₃ alkyl, —X(C₁₋₃ alkyl), —X(C₁₋₃ haloalkyl), —(C₁₋₃ alkyl)OH, —(C₁₋₃ alkyl)CN and C₁₋₃ haloalkyl, or alternatively R⁵ and R⁶ together with the atom to which they are attached form a three to four membered carbocyclic ring optionally substituted with one or more halide, or a four membered heterocyclic ring;
A is selected from the group consisting of C—H, C-halide, and N; and
each X is independently selected from O or S.

2. The compound of claim 1, wherein R¹ and R² are independently selected from the group consisting of H, halide, CN, C₁₋₃ alkyl, —(C₁₋₃ alkyl)CN, —(C₁₋₃ alkyl)OH, and C₁₋₃ haloalkyl, or alternatively R¹ and R² together with the atom to which they are attached form a three to four membered carbocyclic ring optionally substituted with one or more halide, or a four membered heterocyclic ring.

3. The compound of claim 1, wherein R¹ and R² are H.

4. The compound of claim 1, wherein R¹ is H and R² is selected from the group consisting of halide, CN, C₁₋₃ alkyl, —(C₁₋₃ alkyl)CN, —(C₁₋₃ alkyl)OH, and C₁₋₃ haloalkyl.

5. The compound of claim 4, wherein R² is selected from the group consisting of CN, CH₃, CH₂OH, CH₂CN and CH₂F.

6. The compound of claim 1, wherein R¹ and R² are independently selected from the group consisting of halide and C₁₋₃ alkyl.

7. The compound of claim 1, wherein R¹ and R² together with the atom to which they are attached form a three to four membered carbocyclic ring optionally substituted with one or more halide, or a four membered heterocyclic ring.

8. The compound of claim 7, wherein R¹ and R² together with the atom to which they are attached form a ring selected from the group consisting of cyclobutyl, 3-fluoro-cyclobutyl, 2-oxetanyl, and 3-oxetanyl.

9. The compound of claim 1, wherein R³ and R⁴ are independently selected from the group consisting of C₁₋₃ alkyl and —(C₁₋₃ alkyl)OH.

10. The compound of claim 1, wherein R³ and R⁴ are independently selected from the group consisting F, CH₃, CH₂F, CHF₂, CN, OCH₃, SCH₃, OCH₂F, SCH₂F, OCHF₂, SCHF₂, and CH₂CN.

11. The compound of claim 1, wherein R³ and R⁴ together with the atom to which they are attached form a ring selected from the group consisting of cyclobutyl, 3-fluoro-cyclobutyl, 2-oxetanyl, 3-oxetanyl, and cyclopropyl.

12. The compound of claim 1, wherein R⁵ and R⁶ are independently selected from the group consisting of H, halide, C₁₋₃ alkyl, —X(C₁₋₃ alkyl), —X(C₁₋₃ haloalkyl), —(C₁₋₃ alkyl)OH, and C₁₋₃ haloalkyl, or alternatively R⁵ and R⁶ together with the atom to which they are attached form a three to four membered carbocyclic ring optionally substituted with one or more halide, or a four membered heterocyclic ring.

13. The compound of claim 1, wherein R⁵ and R⁶ are H.

14. The compound of claim 1, wherein R⁵ is H and R⁶ is selected from the group consisting of halide, CN, C₁₋₃ alkyl, —X(C₁₋₃ alkyl), —X(C₁₋₃ haloalkyl), —(C₁₋₃ alkyl)CN, —(C₁₋₃ alkyl)OH, and C₁₋₃ haloalkyl.

15. The compound of claim 14, wherein R⁶ is selected from the group consisting of F, CH₃, CH₂F, CHF₂, CN, OCH₃, SCH₃, OCH₂F, SCH₂F, OCHF₂, SCHF₂, and CH₂CN.

16. The compound of claim 1, wherein R⁵ and R⁶ are independently selected from the group consisting of halide and C₁₋₃ alkyl.

17. The compound of claim 1, wherein R⁵ and R⁶ together with the atom to which they are attached form a three to four membered carbocyclic ring optionally substituted with one or more halide, or a four membered heterocyclic ring.

18. The compound of claim 17, wherein R⁵ and R⁶ together with the atom to which they are attached form a ring selected from the group consisting of cyclobutyl, 3-fluoro-cyclobutyl, 2-oxetanyl, 3-oxetanyl, and cyclopropyl.

19. The compound of claim 1, wherein A is selected from the group consisting of C—H, C—Cl, and N.

20. The compound of claim 1, having a structure selected from the group consisting of: [chemical structure] [chemical structure] [chemical structure] [chemical structure] [chemical structure] [chemical structure] [chemical structure] [chemical structure]
or a pharmaceutically acceptable salt thereof.

21. A pharmaceutical composition, comprising a compound of claim 1 and a pharmaceutically acceptable carrier, diluent, excipient or combination thereof.

22. A method of treating a bacterial infection, comprising administering to a subject in need thereof a compound according to claim 1.

23. The method of claim 22, wherein the bacteria is a gram negative bacteria.

24. The method of claim 22, wherein the infection comprises a bacteria that includes Pseudomonas aeruginosa, Pseudomonas fluorescens, Pseudomonas acidovorans, Pseudomonas alcaligenes, Pseudomonas putida, Stenotrophomonas maltophilia, Burkholderia cepacia group, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Acinetobacter anitratis, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtherias, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

25. The method of claim 22, wherein the infection comprises a bacteria that includes Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.
